(19) United States
(12) Patent Application Publication
(10) Pub. No.: US 2009/0100911 A1
(43) Pub. Date: Apr. 23, 2009

(54) Method for producing synthetic resin mold package, alcohol concentration sensor and apparatus for measuring alcohol concentration

(76) Inventors: Toshiaki Kawanishi, Saitama (JP); Shinichi Inoue, Saitama (JP); Takayuki Takahata, Saitama (JP); Kiyoshi Yamagishi, Saitama (JP)

Correspondence Address: Ronald R. Santucci;Frommer Lawrence & Haug, 745 Fifth Avenue, New York, NY, 10151, US

(21) Appl. No.: 10/584,813

(22) PCT Filed: Jan. 12, 2005
(86) PCT No.: PCT/JP2005/000221

§ 371 Date: Jun. 27, 2006

(30) Foreign Application Priority Data
Jan. 13, 2004 (JP) 2004-005614
Jan. 13, 2004 (JP) 2004-005615

(51) Int. Cl. G01N 33/22; H01L 21/56; G01N 27/22

(52) U.S. Cl. 73/61.43; 264/272.13; 73/61.61; 324/686; 324/679

(57) ABSTRACT

Disclosed is a method for producing a synthetic resin mold package at high yield from which package a pair of the surface of an internal device is exposed. A to-be-exposed part of the surface of the internal device composed of an insulating substrate (2) and thin film electrodes (4, 5) and an insulating protective film (6) formed on the substrate is covered with a coating agent (42), and a die pad portion (8) is bonded to the back surface of the internal device. After placing the thus-obtained structure in a mold consisting of a lower mold (46) and an upper mold (48), a pin (50) is inserted into the mold so that the front end of the pin is pressed against the die pad portion (8), thereby keeping the surface of the coating agent (42) pressed against the inner surface of the upper mold (48). Then, a synthetic resin (52) is injected into the mold and cured therein. The thus-obtained resin sealed body is taken out from the mold, and the coating agent (42) is removed from the resin sealed body.

### TECHNICAL FIELD

This invention relates to a method of manufacturing a synthetic resin mold package and, more particularly, to a method of manufacturing a synthetic resin mold package in which a part of the surface of an internal element sealed in the synthetic resin mold of the package is exposed to the outside.

Such a method of manufacturing the synthetic resin mold package can typically find applications in manufacturing an alcohol concentration sensor for measuring the concentration of alcohol such as ethanol and/or methanol contained in gasoline to be used as fuel in an internal combustion engine of an automobile.

The present invention also relates to such an alcohol concentration sensor and an alcohol concentration measuring apparatus using the alcohol concentration sensor.

### BACKGROUND ART

Gasoline, which is a type of fossil fuel, is used in internal combustion engines of automobiles.

However, in view of the circumstance that the production of fossil fuel can fall in the future and that the emission of carbon dioxide is required to be reduced to prevent the earth from warming, it has been studied to mix alcohol such as ethanol or methanol, which is fuel originating from plants, with gasoline so as to be used as fuel for internal combustion engines.

Gasoline and alcohol show respective stoichiometic air-fuel ratios or stoichiometries that are different from each other remarkably. Accordingly, in order to improve the output efficiency of the internal combustion engine with use of the alcohol-gasoline mixture so as to reduce the fuel cost and also reduce the ratio of hydrogen carbide (HC) and carbon monoxide (CO), which are products of incomplete combustion, in exhaust gas, it is necessary to mix air with the alcohol-gasoline mixture at an ideal ratio (that is, to optimize the air-fuel ratio), which varies as a function of the mixing ratio of alcohol relative to gasoline (alcohol concentration), before it is burnt.

Thus, it is preferable to measure the alcohol concentration in gasoline to be used as fuel and control the engine according to the outcome of the measurement. In other words, it is desirable to realize a suitable condition of fuel combustion (the condition of combustion by which the output torque of the internal combustion engine is raised and the rate of producing incomplete combustion products is reduced) by measuring the alcohol concentration in the gasoline actually being supplied to the internal combustion engine and appropriately defining the condition of combustion in the internal combustion engine according to the outcome of the measurement.

Techniques of measuring the alcohol concentration in gasoline and controlling the internal combustion engine according to the outcome of the measurement are disclosed, for example, in JP(A)-4-350550 (Patent Document 1), JP(A)-5-288707 (Patent Document 2) and JP(A)-6-27073 (Patent Document 3).

The sensors for measuring the alcohol concentration in gasoline as disclosed in these Patent Documents are those of the electrostatic capacitance type in which the gasoline to be observed is interposed between a pair of electrodes that produce a capacity and the alcohol concentration is measured by utilizing the fact that the capacity value between the pair of electrodes varies as a function of the alcohol concentration in the gasoline.

The Patent Document 1 describes an alcohol concentration sensor that can advantageously be downsized to show an enhanced performance, wherein a pair of electrodes are formed and separated from each other on the surface of an insulating substrate. According to the patent document, insulating substrates that can preferably be used for such a sensor include those of Al₂O₃ type ceramic and those of steatite type ceramic.

Meanwhile, the techniques described in the above-cited Patent Documents are intended to measure the alcohol concentration in gasoline over a wide range extending between 0% and 100%, so that the change in the capacity value between a pair of electrodes is observed to measure the alcohol concentration that is found in such a wide range.

While it may be ideal to control an internal combustion engine in correspondence to the entire region of such a wide range, it is actually difficult to satisfactorily control the engine only by controlling the air-fuel ratio because, in reality, the design of the engine may have to be changed to achieve such a perfect controllability.

Patent Document 1: JP(A)-4-350550

Patent Document 2: JP(A)-5-288707

Patent Document 3: JP(A)-6-27073

### DISCLOSURE OF THE INVENTION

### Problems to be Solved by the Invention

An insulating layer is formed to cover a major part of the electrodes formed on a substrate in the alcohol concentration sensor described in the above-cited Patent Document 1. However, no cover is provided on the parts of the surface of the substrate where the electrode pads are formed, the rear surface and the lateral end faces.

When downsizing an alcohol concentration sensor, it is desirable to improve the easiness of handling it and raise the strength and durability of the sensor. Particularly, it is highly desirable to avoid a situation where the electrodes are peeled off from the substrate because of a reduced adhesion of the electrodes relative to the substrate that occurs as a result of penetration of alcohol-containing gasoline. For this purpose, it is preferable to seal the substrate, on which the electrodes are formed, with a synthetic resin mold to produce a mold package. In the case of an alcohol concentration sensor, it is necessary to partially seal it with resin in such a way that the substrate surface on which the electrodes are formed is exposed in order to bring the electrodes formed on the substrate into contact with or close to the alcohol-containing gasoline, which is the liquid to be observed. Manufacture of such partially sealed mold packages can give rise to defective products because resin can remain on the exposed surface. Then, it is difficult to manufacture such mold packages at a high yield, preventing production of such defective products.

Such difficulty arises not only when manufacturing alcohol concentration sensors but also when manufacturing synthetic resin mold packages exposing at least a part of the surface of the internal element sealed in the inside regardless of the type of the sealed internal element.

Thus, it is an object of the present invention to manufacture synthetic resin mold packages exposing a part of the surface of the internal element, which may typically be an alcohol concentration sensor as described above, at a high yield.

The range of alcohol concentration in gasoline that can improve the output efficiency and reduce the incomplete combustion products in exhaust gas by controlling the air-fuel ratio of an internal combustion engine adapted to use alcohol-mixed gasoline without significantly changing the design of the traditional internal combustion engine is typically between 0 and 5%. In other words, an engine that is designed to be driven by combusting pure gasoline can be operated well by using alcohol-mixed gasoline of such a relatively low alcohol concentration when the air-fuel ratio is controlled appropriately.

Therefore, it is another object of the present invention to make it possible to precisely control the air-fuel ratio in an internal combustion engine adapted to use alcohol-mixed gasoline by accurately measuring the alcohol concentration in gasoline within such a low alcohol concentration range as cited above and, especially to provide an alcohol concentration sensor for precisely controlling the air-fuel ratio.

### Means for Solving the Problem

In order to attain the above objects, according to the present invention, there is provided a method of manufacturing a synthetic resin mold package by sealing an internal element with synthetic resin so as to expose at least a part of the surface of the internal element, comprising:

a coating step of coating a part to be exposed of the surface of the internal element with a coating agent;

a bonding step of bonding a die pad portion to a rear surface of the internal element;

an arranging step of arranging a structure obtained by way of the coating step and the bonding step in a mold;

a pressing step of inserting a pin into the mold after the arranging step to make the front end thereof abut on the die pad portion and pressing a surface of the coating agent against the inner surface of the mold;

an injecting/setting step of injecting synthetic resin into the mold and setting the synthetic resin after the pressing step;

a taking-out step of taking out a resin-sealed body obtained by way of the injecting/setting step from the mold; and

a removing step of removing the coating agent from the resin-sealed body.

In an aspect of the present invention, the coating agent is photoresist, and the coating agent is removed from the resin-sealed body by immersing the resin-sealed body in a solvent in the removing step. In an aspect of the present invention, the internal element is formed by forming an electrically conductive thin film on a surface of an insulating substrate, and the electrically conductive thin film extends from a part to be exposed of a surface of the internal element to a part other than the part to be exposed and has an electrode pad section formed in the part other than the part to be exposed. In an aspect of the present invention, the electrically conductive thin film is covered by an insulating protective film in the part to be exposed. In an aspect of the present invention, the electrically conductive thin film includes a pair of thin film electrodes arranged to produce an electrostatic capacitance. In an aspect of the present invention, a specific dielectric constant of the insulating substrate is not higher than 5. In an aspect of the present invention, the die pad portion is connected to a lead section to form a lead frame in the bonding step, the electrode pad section and the lead section are electrically connected after the bonding step and before the arranging step, and the lead frame is cut and the die pad portion is separated from the lead section after the taking-out step.

In order to attain the above objects, according to the present invention, there is also provided an alcohol concentration sensor of an electrostatic capacitance type for measuring an alcohol concentration in gasoline mixed with alcohol, comprising:

an insulating substrate; and

a pair of thin film electrodes arranged on a surface of the insulating substrate to produce an electrostatic capacitance,

wherein the insulating substrate is made of a material showing a specific dielectric constant of not higher than 5.

In an aspect of the present invention, the insulating substrate has a thickness between 200 and 1000 μm. In an aspect of the present invention, the pair of thin film electrodes have a thickness between 0.01 and 0.8 μm. In an aspect of the present invention, each of the pair of thin film electrodes is at least partly covered by an insulating protective film. In an aspect of the present invention, the insulating protective film is made of a material showing a specific dielectric constant of not higher than 5. In an aspect of the present invention, the insulating protective film has a thickness between 0.4 and 1 μm.

In an aspect of the present invention, the alcohol concentration sensor further comprises a pair of lead-out electrodes connected respectively to the pair of thin film electrodes; and a resin mold for sealing connection ends of the lead-out electrodes connected to the thin film electrodes and a part of the insulating substrate, wherein the resin mold exposes to the outside at least a part of the surface of the insulating substrate with the thin film electrodes formed thereon.

In order to attain the above objects, according to the present invention, there is also provided an alcohol concentration measuring apparatus, comprising: an oscillation circuit including the pair of thin film electrodes of an alcohol concentration sensor as claimed in claim 8; and a processing section for computationally determining the alcohol concentration according to an oscillation frequency of the oscillation circuit.

In an aspect of the present invention, the processing section computationally determines the alcohol concentration using a calibration curve. In an aspect of the present invention, the calibration curve shows a relationship between the alcohol concentration and the oscillation frequency of the oscillation circuit within a range of alcohol concentration between 0 and 5% and a corresponding range of the oscillation frequency of the oscillation circuit.

### Effects of the Invention

Thus, with a method of manufacturing a synthetic resin mold package according to the present invention, the part to be exposed of the surface of an internal element is coated with a coating agent, and a die pad portion is bonded to the rear surface of the internal element. Then, the obtained structure is arranged in a mold, and subsequently a pin is inserted into the mold until the front end of the pin abut on the die pad portion. Then, the surface of the coating agent is pressed against the inner surface of the mold, and the pressed condition is maintained. Thereafter, synthetic resin is injected into the mold and set. The obtained resin-sealed body is taken out from the mold and the coating agent is removed from the resin-sealed body. Thus, it is possible to manufacture synthetic resin mold packages that expose a part of the surface of the internal element thereof to the outside with ease at a high yield.

An insulating substrate on the surface of which a pair of thin film electrodes are arranged to produce an electrostatic capacitance is made of a material showing a specific dielectric constant of not higher than 5 and used for an electrostatic capacitance type alcohol concentration sensor according to the present invention. Thus, it is possible to accurately measure the alcohol concentration within a relatively low alcohol connection range with an enhanced degree of sensitivity. Then, it is possible to precisely control the air-fuel ratio in an internal combustion engine adapted to use alcohol-mixed gasoline.

### BRIEF DESCRIPTION OF THE DRAWINGS

FIG. 1 is a schematic perspective view of an embodiment of alcohol concentration sensor manufactured by the present invention;

FIG. 2 is a schematic cross-sectional view of the alcohol concentration sensor of FIG. 1;

FIG. 3 is a schematic perspective view illustrating an insulating substrate and thin film electrodes of the alcohol concentration sensor of FIG. 1;

FIG. 4 is a schematic cross-sectional view of an alcohol concentration sensor, illustrating a step of manufacturing it;

FIG. 5 is a schematic cross-sectional view of an alcohol concentration sensor, illustrating a step of manufacturing it;

FIG. 6 is a schematic cross-sectional view of an alcohol concentration sensor, illustrating a step of manufacturing it;

FIG. 7 is a plan view of an alcohol concentration sensor, illustrating a step of manufacturing it;

FIG. 8 is a schematic cross-sectional view of an alcohol concentration sensor, illustrating a step of manufacturing it;

FIG. 9 is a schematic cross-sectional view of an alcohol concentration sensor, illustrating a step of manufacturing it;

FIG. 10 is a schematic cross-sectional view of an alcohol concentration sensor, illustrating a step of manufacturing it;

FIG. 11 is a schematic cross-sectional view of an alcohol concentration sensor, illustrating a step of manufacturing it;

FIG. 12 is a schematic cross-sectional view of an alcohol concentration sensor, illustrating a step of manufacturing it;

FIG. 13 is a schematic illustration of an embodiment of alcohol concentration measuring apparatus;

FIG. 14 is a graph illustrating the characteristics of the rate of change of the oscillation frequency of the oscillation circuit relative to the change of the ethanol concentration; and

FIG. 15 shows an alcohol concentration sensor arranged at a gasoline flow path,

wherein reference numeral 2 denotes an insulating substrate, 4,5 thin film electrode, 4a, 5a pad section of the thin film electrode, 6 insulating protective film, 8 die pad portion of lead frame, 10, 11 lead section of the lead frame (lead-out electrodes), 12 bonding wire, 14 resin mold, 20 alcohol concentration sensor, 22 oscillation circuit, 26 microcomputer, 28 output buffer circuit, VDD input of the oscillation circuit, OUT output of the oscillation circuit, ER1, ER2 resistance element, EC capacitance element, 30 measuring section housing main body, 31 measuring section housing lid body, 32 fuel tank side pipe, 34 internal combustion engine side pipe, 42 coating agent, 44 lead frame, lower mold, 46a pin insertion hole, 48 upper mold, 50 pin, and 52 denotes a synthetic resin.

### BEST MODE FOR CARRYING OUT THE INVENTION

Now, the present invention will be described in greater detail by referring to the accompanying drawings that illustrate preferred embodiments of the present invention. While the embodiment described below relates to a synthetic resin mold package for an alcohol concentration sensor, a synthetic resin mold package according to the present invention is by no means limited thereto and may apply to any internal elements having a broad scope of applications.

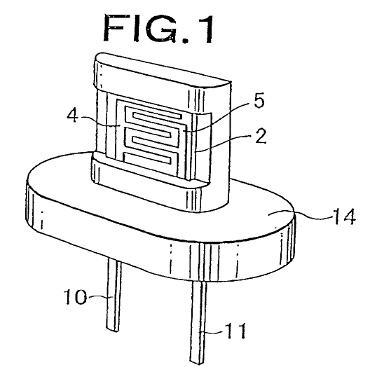

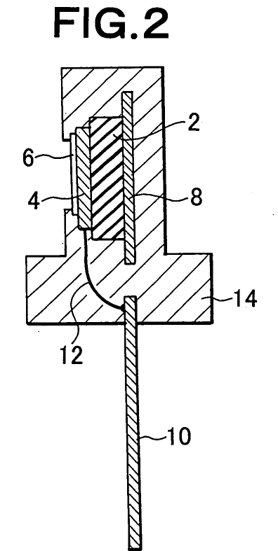

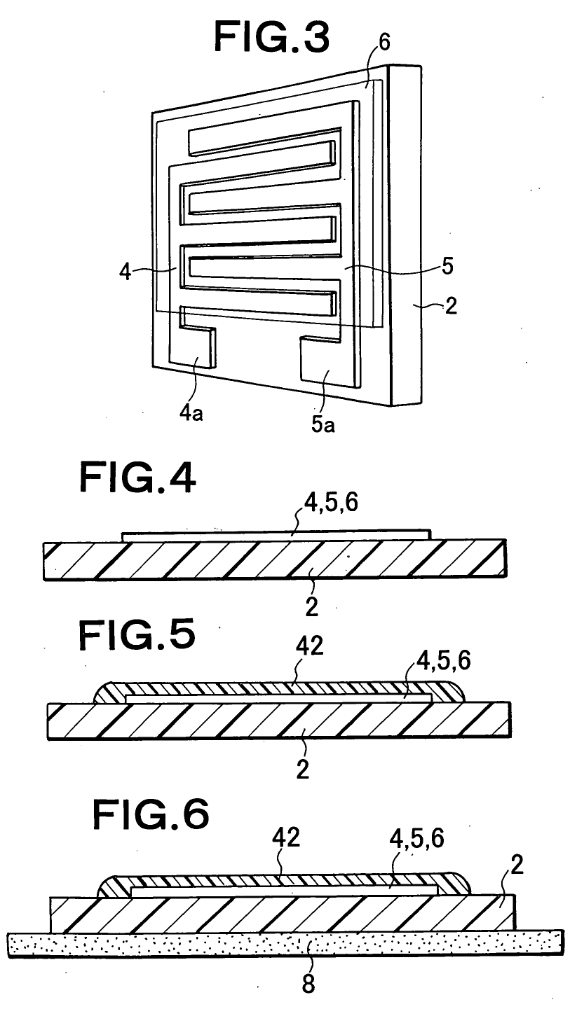

Firstly, an embodiment of alcohol concentration sensor that is manufactured by this embodiment will be described. FIG. 1 is a schematic perspective view of the embodiment of alcohol concentration sensor and FIG. 2 is a schematic cross-sectional view of the embodiment, while FIG. 3 is a schematic perspective view illustrating the insulating substrate and the thin film electrodes of the embodiment of alcohol concentration sensor.

In this embodiment, a pair of thin film electrodes 4, 5 and an insulating protective film 6 that is formed to cover the thin film electrodes are formed on one of the main surfaces (front surface) of an insulating substrate 2.

The insulating substrate 2 is made of a material showing a specific dielectric constant of not higher than 5 and typically having a thickness between 200 and 1000 μm. Materials showing a specific dielectric constant of not higher than 5 that can be used for the insulating substrate 2 include Pyrex (trademark) glass, fused quartz and synthetic resins such as Teflon (trademark), nylon, polyethylene, polystyrene, polymethyl methacrylate and bakelite. The purpose of using an insulating substrate 2 made of a material showing a specific dielectric constant of not higher than 5 will be described hereinafter.

The thin film electrodes 4, 5 are made of a highly corrosion-resistant electric conductor selected from aluminum, gold, silver, copper, titanium, nickel, chromium and alloys of any of them and typically have a thickness between 0.01 and 0.8 μm. As illustrated, the thin film electrodes 4, 5 are arranged so as to show an interdigital pattern. Alternatively, the thin film electrodes 4, 5 may be realized in the form of double winding as described in the above cited Patent Document 1. As described in the Patent Document 1, when the electrodes 4, 5 are realized as a pair of patterned thin film electrodes in the same plane, the distance between the electrodes practically does not change if the insulating substrate is bent or otherwise deformed, to thereby show excellent capacity stability. The thin film electrodes 4, 5 may typically be obtained by forming an electrically conductive film on the surface of the insulating substrate 2 by sputtering and subjecting the electric conductive film to a patterning operation using photolithography. The thin film electrodes 4, 5 are provided respectively with pad sections 4a, 5a at an end so as to be connected to lead-out electrodes which will be described in greater detail hereinafter.

The insulating protective film 6 is provided to protect the thin film electrodes 4, 5 against chemical damages that can be caused by alcohol-containing gasoline, which is the liquid to be measured, and to prevent any electric current from flowing between the thin film electrodes 4, 5 by way of alcohol-containing gasoline, especially the moisture contained therein. Examples of materials that can be used for the insulating protective film 6 include electric insulators such as SiO₂, Si₃N₄ and Al₂O₃. Note that the insulating protective film 6 is not formed on the pad sections 4a, 5a of the thin film electrodes 4, 5. The thickness of the insulating protective film 6 is typically between 0.4 and 1 μm. The detection sensitivity of the alcohol concentration sensor for detecting the specific dielectric constant of alcohol-containing gasoline is too low when the insulating protective film 6 is too thick. Therefore, the insulating protective film 6 is preferably as thin as possible from this point of view. On the other hand, pin holes can be produced to make it hard to achieve the intended effect when the insulating protective film 6 is too thin. Therefore, the insulating protective film 6 is preferably as thick as possible from this point of view. Preferably, the material of the insulating protective film 6 shows a specific dielectric constant not higher than 5 as in the case of the material of the insulating substrate 2. The purpose of using an insulating protective film 6 made of a material showing a specific dielectric constant of not higher than 5 will be described hereinafter. The insulating protective film 6 can typically be formed by sputtering. It is not necessary to use the insulating protective film 6 when the alcohol-containing gasoline does not practically contain electrically conductive impurities. When the thickness of the insulating protective film 6 is sufficiently small (e.g., not more than ⅕) relative to the distance between the oppositely disposed thin film electrodes 4, 5, the specific dielectric constant of the insulating protective film 6 does not influence the detection sensitivity for the specific dielectric constant of alcohol-containing gasoline significantly and therefore the insulating protective film 6 may be formed by using a material showing a specific dielectric constant that exceeds 5.

The insulating substrate 2 is bonded at the rear surface thereof to die pad portion 8 of a lead frame. On the other hand, the pad sections 4a, 5a of the thin film electrodes are connected respectively to lead sections (lead-out electrodes) 10, 11 of the lead frame by means of bonding wires 12. The connection ends of the lead-out electrodes 10, 11 connected to the thin film electrodes (the ends of the lead-out electrodes 10, 11 where the bonding wires 12 are connected), part of the insulating substrate 2, the die pad portion 8 and the bonding wires 12 are sealed by a resin mold 14. The resin mold 14 exposes the part of the surface of the insulating substrate 2 where the thin film electrodes 4, 5 are formed so that the thin film electrodes 4, 5 can be placed close to the alcohol-containing gasoline to be measured for the alcohol concentration thereof by way of the insulating protective film 6.

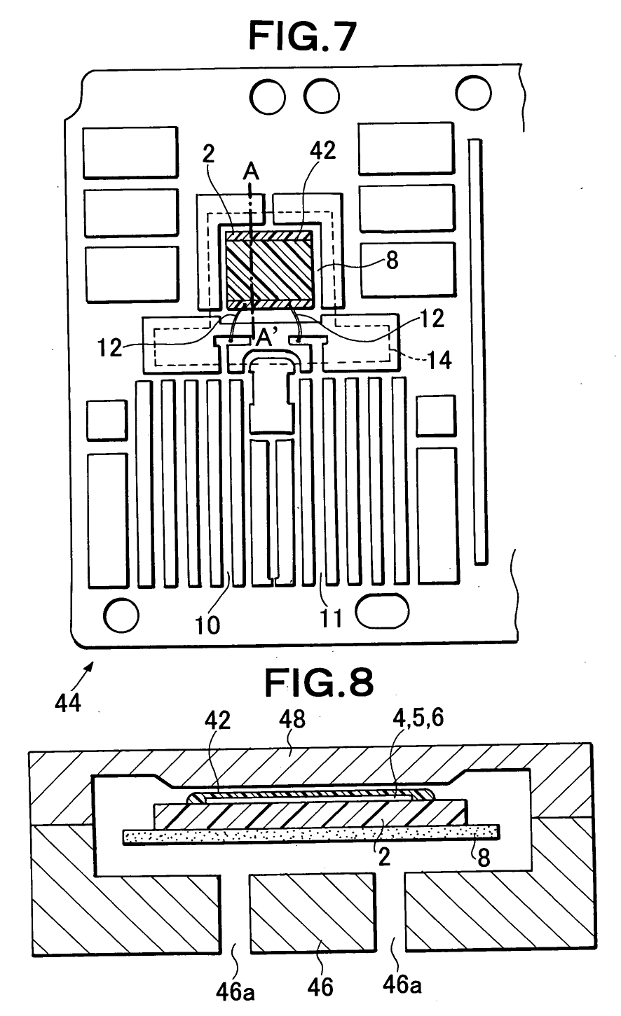

Now, an embodiment of method of manufacturing an alcohol concentration sensor and hence a synthetic resin mold package according to the present invention will be described below by referring to FIGS. 4 through 12. FIGS. 4 through 6 and FIGS. 8 through 12 are schematic cross-sectional views of an alcohol concentration sensor, illustrating different steps of manufacturing it, while FIG. 7 is a plan view of the alcohol concentration sensor, illustrating a step of manufacturing it. Note that FIGS. 4 through 6 and FIGS. 8 through 12 are cross-sectional views taken along line A-A′ in FIG. 7.

Firstly, as shown in FIG. 4, thin film electrodes 4, 5 that are electrically conductive thin films and an insulating protective film 6 are formed on the surface of an insulating substrate 2. In the instance of FIGS. 4 through 6 and FIGS. 8 through 12, the thin film electrodes 4, 5 and the insulating protective film 6 are integrally illustrated for the purpose of simplicity. As a result, an internal element or internal device is formed for the purpose of the present invention. The thin film electrodes 4, 5 extend respectively from the part of the surface of the internal element to be exposed to the outside to a part other than the part to be exposed and have the electrode pad sections 4a, 5a formed in the part of the surface other than the part of the surface to be exposed (to-be-exposed part). The thin film electrodes 4, 5 are covered by the insulating protective film 6 in the part of the surface to be exposed.

Then, a coating step is conducted. As shown in FIG. 5, the part of the surface of the internal element to be exposed is coated with a coating agent 42. The coating agent 42 is preferably photoresist because it can easily and conveniently be produced with a flat surface and a predetermined pattern.

Thereafter, a bonding step is conducted. As shown in FIG. 6, a die pad portion 8 is bonded to the rear surface of the internal element (and hence the rear surface of the insulating substrate 2) by means of a bonding agent. In this step, the die pad portion 8 is connected to the lead sections 10, 11 and other sections to form a lead frame 44 as shown in FIG. 7.

After the bonding step, the pad sections 4a, 5a of the thin film electrodes 4, 5 and the lead sections 10, 11 are electrically connected respectively by way of the above-described bonding wires 12 as shown in FIG. 7.

Subsequently, an arranging step is conducted. In this step, the structure obtained as a result of the coating step and the bonding step is arranged in a mold as shown in FIG. 8. The mold includes a lower mold 46 and an upper mold 48 and the top surface of the lower mold 46 and the bottom surface of the upper mold 48 operate as molding surfaces. The lower mold 46 is provided with pin insertion holes 46a that are vertical through holes.

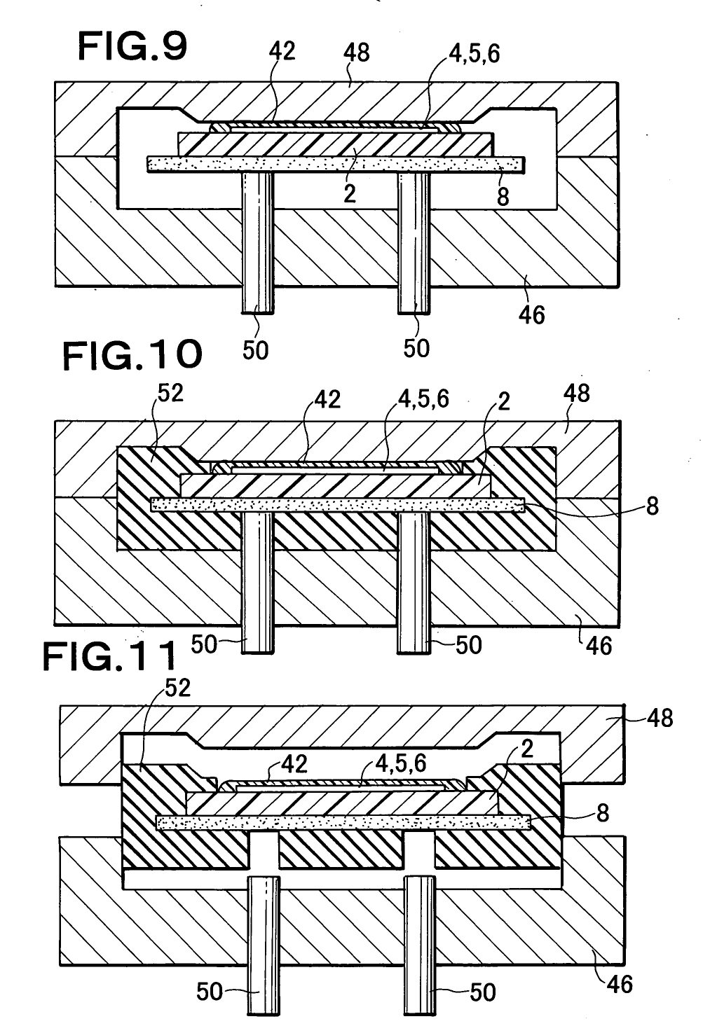

Then, a pressing step is conducted. In this step, pins 50 are inserted into the mold by way of the respective pin insertion holes 46a until the front ends thereof abut on the die pad portion 8 and the surface (top surface) of the coating agent 42 is pressed against the molding surface that is an inner surface of the mold, or the bottom surface of the upper mold 48 as shown in FIG. 9. The pins 50 are kept pressing the internal element during the step.

Thereafter, an injecting/setting step is conduced. In this step, synthetic resin 52 is injected into the mold and set as shown in FIG. 10. The above-described resin mold 14 is formed by the set synthetic resin 52. Thus, a resin-sealed body where the internal element is sealed by the resin mold is produced. Since the surface of the coating agent 42 and the bottom surface of the upper mold 48 are held in tight contact with each other, no synthetic resin 52 flows between them.

Subsequently, a taking-out step is conducted. In this step, the mold is opened and the above-described resin-sealed body is taken out from the mold as shown in FIG. 11.

After the taking-out step, as shown in FIG. 7, all the parts of the lead frame 44 located outside the resin mold 14 are removed by cutting except the lead sections 10, 11. As a result, the die pad portion 8 is separated from the lead sections 10, 11.

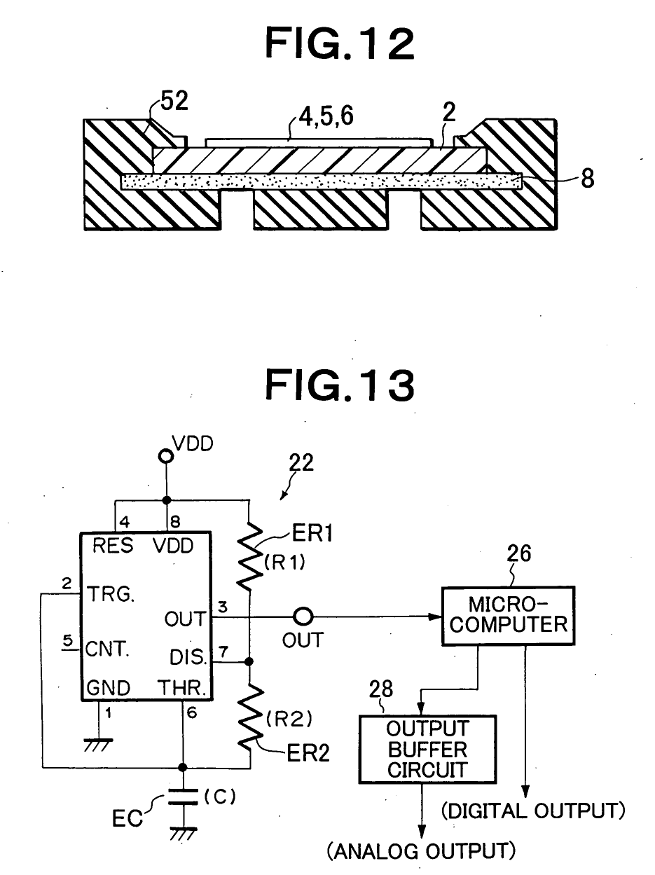

Thereafter, a removing step is conducted. In this step, the above-described coating agent 42 is removed from the resin-sealed body, from which the unnecessary part of the lead frame 44 has been cut away, as shown in FIG. 12. In this removing step, the coating agent, which may typically be photoresist, can be removed from the resin-sealed body by immersing the resin-sealed body in an organic solvent such as acetone.

As a result of using the above-described manufacturing method, practically no synthetic resin 52 remains on the surface of the internal element coated with the coating agent. Therefore, it is possible to manufacture such alcohol concentration sensors at a high yield.

FIG. 13 is a schematic illustration of an embodiment of alcohol concentration measuring apparatus by using an alcohol concentration sensor according to the present invention and having a configuration as described above. The apparatus comprises an oscillation circuit 22 and a microcomputer 26. The microcomputer 26 operates as a processing section for computationally determining the alcohol concentration according to the frequency of the output signal of the oscillation circuit 22, or the oscillation frequency of the oscillation circuit 22. The input VDD of the oscillation circuit 22 is typically equal to 5V and the output OUT thereof is determined as a function of the resistances R1, R2 of resistance elements ER1, ER2 and the capacitance C of capacitance element EC. The capacitance element EC is formed by using the thin film electrodes 4, 5 of an alcohol concentration sensor according to the invention that is described above by referring to FIGS. 1 through 12. The capacitance C of the capacitance element EC is influenced by the specific dielectric constant of the substance interposed between the pair of thin film electrodes 4, 5. In this embodiment, when a voltage is applied between the pair of thin film electrodes 4, 5, some of the electric lines of force formed between the pair of thin film electrodes 4, 5 pass through alcohol-containing gasoline while the others pass through the insulating substrate 2.

The pulse width T of the output signal of the oscillation circuit 22 (which is the reciprocal of the oscillation frequency f) shows a relationship as defined by the formula below with C, R1 and R2.

1/T=f=1.44/[C(R1+2·R2)]

If the alcohol concentration (e.g., ethanol concentration) in gasoline, the specific dielectric constant of the insulating substrate 2, the specific dielectric constant of gasoline, the specific dielectric constant of alcohol (e.g., ethanol) and the dielectric constant of vacuum are respectively α, εsub, εr[g], εr[a] and ε0 and the area of the electrodes and the distance between the electrodes are respectively S and d when it is assumed that the capacitance element EC is comprised of parallel plates, the capacitance C of the capacitance element EC is expressed by the formula below when there is no insulating protective film 6.

C=ε0(S/d)(εr[g](1−α)+εr[a]α+εsub)

Therefore, if the electrostatic capacitance of gasoline is C[g] when the alcohol concentration is 0 and C[a] when the alcohol concentration is 1, the electrostatic capacitance changes between the above two values at a rate as expressed by the formula below.

(C[a]−C[g])/C[g]=(εr[a]−εr[g])/(εr[g]+εsub)

From the above formula, it will be seen that the rate of change of the electrostatic capacitance of the capacitance element EC is improved when a material showing a low specific dielectric constant is used for the insulating substrate 2.

When an insulating protective film 6 is provided, the relationship may become more complex, but nevertheless the rate of change of the electrostatic capacitance of the capacitance element EC is also improved by using an insulating protective film 6 that shows a low specific dielectric constant like the insulating substrate 2.

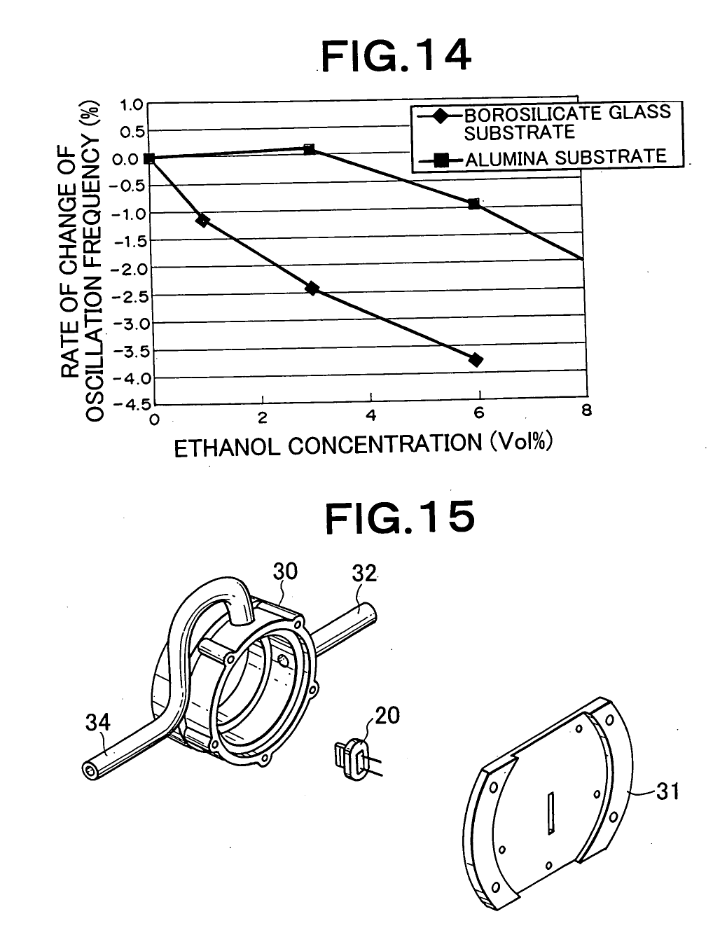

FIG. 14 is a graph illustrating the characteristics of the rate of change of the oscillation frequency f of the output signal of the oscillation circuit 22 relative to the change in the ethanol concentration when ethanol was used as alcohol (the rate of change when the ethanol concentration 0 is used as reference value) in an experiment. In this experiment, an insulating substrate 2 made of Pyrex [trademark] glass (borosilicate glass) showing a specific dielectric constant of 4.84 (embodiment of the present invention) and an insulating substrate 2 made of alumina showing a specific dielectric constant of 9.34 to 11.54 (comparative example), both having a thickness of 250 μm, were used for comparison. The thin film electrodes 4, 5 had a thickness of 0.4 μm and the distance between the oppositely disposed thin film electrodes 4, 5 was 10 μm. The insulating protective film 6 showed a specific dielectric constant of 4 and had a thickness of 0.4 μm (or 1/25 of the distance between the oppositely disposed thin film electrodes 4, 5).

From FIG. 14, it will be seen that the embodiment of the present invention showed a high rate of change of the oscillation frequency of the oscillation circuit 22 when the ethanol concentration is not higher than 5% so that the ethanol concentration was measured with an enhanced degree of sensitivity because of the high rate of change.

The output of the oscillation circuit 22 is input to the microcomputer 26, which then computationally determines the rate of change of the oscillation frequency with use of an oscillation frequency value at the ethanol concentration of 0 stored in the memory as reference value, and then determines the ethanol concentration by referring to the calibration curve also stored in the memory. The calibration curve is obtained by observing samples of gasoline showing known ethanol concentrations like the one shown in FIG. 14 and stored in the memory. Alternatively, a calibration curve prepared by using not the rate of change of the oscillation frequency but the frequency values itself may be used. In this case, the microcomputer 26 is not required to computationally determine the rate of change of the oscillation frequency.

Then, a signal that indicates the obtained ethanol concentration value is output to an output buffer circuit 28 shown in FIG. 13 by way of a D/A converter (not shown) and then output as analog output to a main computer (ECU) (not shown) that is adapted to control the combustion of the engine of the automobile where the alcohol concentration measuring apparatus is mounted. It is possible to realize a suitable condition of combustion that corresponds to the alcohol concentration in the gasoline actually fed for combustion (a condition that raises the output torque of the internal combustion engine and reduce the amount of the incomplete combustion products in exhaust gas) for the engine as the ECU defines such a condition of combustion according to the signal indicating the ethanol concentration value input to it.

On the other hand, the signal indicating the ethanol concentration value can be taken out as digital output, whenever necessary, so that it may be input to equipment that operates to display the ethanol concentration value, output an alarm and/or does other operations.

FIG. 15 shows an alcohol concentration sensor that is provided with a gasoline flow path. A measuring section housing that comprises a measuring section housing main body 30 and a measuring section housing lid body 31 is arranged between the fuel tank side pipe 32 that operates as supply path for supplying alcohol-containing gasoline from the fuel tank to the internal combustion engine and the internal combustion engine side pipe 34. The lid body 31 is fitted to the main body 30 and the alcohol concentration sensor 20 is fitted to the inside of the lid body 31. The lead-out electrodes 10, 11 of the sensor extend to the outside of the lid body 31 and connected to a circuit substrate (not shown) fitted to the outer surface of the lid body 31. An oscillation circuit 22 is formed in or mounted on the circuit substrate and, if necessary, a microcomputer 26, an output buffer circuit 28 and other elements are additionally formed in or mounted on the circuit substrate.

1. A method of manufacturing a synthetic resin mold package by sealing an internal element with synthetic resin so as to expose at least a part of the surface of the internal element, comprising:
a coating step of coating a part to be exposed of the surface of the internal element with a coating agent;
a bonding step of bonding a die pad portion to a rear surface of the internal element;
an arranging step of arranging a structure obtained by way of the coating step and the bonding step in a mold;
a pressing step of inserting a pin into the mold after the arranging step to make the front end thereof abut on the die pad portion and pressing a surface of the coating agent against the inner surface of the mold;
an injecting/setting step of injecting synthetic resin into the mold and setting the synthetic resin after the pressing step;
a taking-out step of taking out a resin-sealed body obtained by way of the injecting/setting step from the mold; and
a removing step of removing the coating agent from the resin-sealed body.

2. The method of manufacturing a synthetic resin mold package as claimed in claim 1, wherein the coating agent is photoresist, and the coating agent is removed from the resin-sealed body by immersing the resin-sealed body in a solvent in the removing step.

3. The method of manufacturing a synthetic resin mold package as claimed in claim 1, wherein the internal element is formed by forming an electrically conductive thin film on a surface of an insulating substrate, and the electrically conductive thin film extends from a part to be exposed of a surface of the internal element to a part other than the part to be exposed and has an electrode pad section formed in the part other than the part to be exposed.

4. The method of manufacturing a synthetic resin mold package as claimed in claim 3, wherein the electrically conductive thin film is covered by an insulating protective film in the part to be exposed.

5. The method of manufacturing a synthetic resin mold package as claimed in claim 3, wherein the electrically conductive thin film includes a pair of thin film electrodes arranged to produce an electrostatic capacitance.

6. The method of manufacturing a synthetic resin mold package as claimed in claim 5, wherein a specific dielectric constant of the insulating substrate is not higher than 5.

7. The method of manufacturing a synthetic resin mold package as claimed in claim 3, wherein the die pad portion is connected to a lead section to form a lead frame in the bonding step, the electrode pad section and the lead section are electrically connected after the bonding step and before the arranging step, and the lead frame is cut and the die pad portion is separated from the lead section after the taking-out step.

8. An alcohol concentration sensor of an electrostatic capacitance type for measuring an alcohol concentration in fuel for internal combustion engine mixed with alcohol, comprising:
an insulating substrate; and
a pair of electrodes arranged on a surface of the insulating substrate to produce an electrostatic capacitance,
wherein the insulating substrate is made of a material showing a specific dielectric constant of not higher than 5.

9. The alcohol concentration sensor as claimed in claim 8, wherein the insulating substrate has a thickness between 200 and 1000 μm.

10. The alcohol concentration sensor as claimed in claim 8, wherein the pair of electrodes have a thickness between 0.01 and 0.8 μm.

11. The alcohol concentration sensor as claimed in claim 8, wherein each of the pair of electrodes is at least partly covered by an insulating protective film.

12. The alcohol concentration sensor as claimed in claim 11, wherein the insulating protective film is made of a material showing a specific dielectric constant of not higher than 5.

13. The alcohol concentration sensor as claimed in claim 11, wherein the insulating protective film has a thickness between 0.4 and 1 μm.

14. The alcohol concentration sensor as claimed in claim 8, further comprising a pair of lead-out electrodes connected respectively to the pair of; electrodes and a resin mold for sealing connection ends of the lead-out electrodes connected to the electrodes and a part of the insulating substrate, wherein the resin mold exposes to the outside at least a part of the surface of the insulating substrate with the electrodes formed thereon.

15. An alcohol concentration measuring apparatus, comprising: an oscillation circuit including the pair of electrodes of an alcohol concentration sensor as claimed in claim 8; and a processing section for computationally determining the alcohol concentration according to an oscillation frequency of the oscillation circuit.

16. The alcohol concentration measuring apparatus as claimed in claim 15, wherein the processing section computationally determines the alcohol concentration using a calibration curve.

17. The alcohol concentration measuring apparatus as claimed in claim 15, wherein the calibration curve shows a relationship between the alcohol concentration and the oscillation frequency of the oscillation circuit within a range of alcohol concentration between 0 and 5% and a corresponding range of the oscillation frequency of the oscillation circuit.

18. The alcohol concentration sensor as claimed in claim 1, wherein the fuel for internal combustion engine is gasoline.

19. The alcohol concentration sensor as claimed in claim 1, wherein the electrodes are thin film electrodes.
